(19) United States
(12) Patent Application Publication
(10) Pub. No.: US 2013/0131535 A1
(43) Pub. Date: May 23, 2013

(54) BRAINWAVE CONTROL SYSTEM AND METHOD OPERABLE THROUGH TIME DIFFERENTIAL EVENT-RELATED POTENTIAL

(71) Applicants: Kuang-Tien Sun, Tainan City (TW); Tzu-Wei Huang, Hsinchu City (TW); Min-Chi Chen, Kaohsiung City (TW); Hsu Sun, Tainan City (TW); Chia-Lin Sun, Tainan City (TW)

(72) Inventors: Kuang-Tien Sun, Tainan City (TW); Tzu-Wei Huang, Hsinchu City (TW); Min-Chi Chen, Kaohsiung City (TW); Hsu Sun, Tainan City (TW); Chia-Lin Sun, Tainan City (TW)

(21) Appl. No.: 13/301,459

(22) Filed: Nov. 21, 2011

(51) Int. Cl. A61B 5/0476

(52) U.S. Cl. 600/544

(57) ABSTRACT

A brainwave control system and method operable through time differential event-related potential includes a brainwave capturing unit attached to a user's head, a brainwave signal processing apparatus and a display unit to display at least two sets of stimuli. The brainwave signal processing apparatus includes a signal processor connected to the brainwave capturing unit and a central processor. The signal processor converts brainwave signals generated by the user after having received a set of short time interval stimuli to digital signals. The central processor performs analysis and generates identification results and executes control commands accordingly. Thus user can rapidly and accurately execute his requirements of operation control so as to achieve non-contact operation control with improved usability and practicality.

### FIELD OF THE INVENTION

The present invention relates to a brainwave control system and method and particularly to a brainwave control system and method operable through time differential event-related potential.

### BACKGROUND OF THE INVENTION

With developments and advances in information technology industries, a growing number of people and enterprises in recent years have devoted in developing more convenient non-contact operation systems and methods, aiming to replace the conventional manual control methods that rely on remote controllers, mouse or joysticks. Among them eye tracking control and brainwave control have greater applicability and can meet most humanized requirements.

Development of eye tracking operating control is based on the concept of eye tracker. At present eye tracking control apparatus have been developed. They employ infrared ray of a video camera to capture eyeball images and process image signals through software. Next, eyeball moving direction and pupil contraction and dilatation are identified; then, eyeball looking position, moving speed and distance and pupil alteration information can be calculated on the basis of the above gathered data to perform operation control functions such as data entry and the like. They still have drawbacks. For instance, users have to maintain eyeball focus during execution of operation control to avoid system identification error. When used in a prolonged period of time fatigue of users' visual sense occurred and results in a heavy spiritual burden.

Brainwave control mainly can be divided into four types of operation control methods. A first type uses an EEG cap (electroencephalography cap) attached to a user's head to receive brainwave signals, then the brainwave signals are compared with a pre-trained and stored brainwave characteristics database (e.g., spectrums of brainwave signals); the mating brainwave characteristics are analyzed and commands are executed accordingly. Such a technique has disadvantages, e.g. the user has to be trained in advance before performing the brainwave control operation to avoid operational inaccuracy, and user's brainwave characteristics have to be saved during training to form the brainwave characteristics database to facilitate comparison and analysis. Based on a long period of experiments, the aforesaid operation control method relies on user's spontaneity to generate the required brainwave signals to the system for analysis, but the generated brainwaves have a wide variety of signals that induces a low accuracy and cannot be used to perform an accurate operation control. It still cannot meet actual application requirements.

A second type of brainwave operation control method resolves the problem of requiring pre-training of users. It is integrated with eye tracking control and provides a plurality of stimuli corresponding to different control commands sent to the users. The users do not need training. By merely focusing the visual sense on a target signal and the target signal causes optical nerve to generate a visual muscle potential via eyeball movements or blinks; a small electric wave alteration is captured and analyzed to control events. However, users' concentration directly affects the success rate of the operation control. Moreover, such a method also cannot eliminate system's faulty interpretation caused by human's intrinsic blink motion (or called blink reflex in a medical term). It still leaves a lot of problems to be improved.

A third type provides an operation control method by integrating brainwave and visual sense, such as U.S. Pat. No. 7,338,171 which discloses a method and apparatus for visual drive control. It provides a plurality of images simultaneously on a display screen at the same frequency but flickering in different fashions to be watched by a user; user's brainwave signals triggered by stimulation of those images are captured; the brainwave signals are processed with similarity analysis to confirm a maximum similarity value between the flickering images and brainwave signals; when the maximum similarity value exceeds a predefined threshold, a control signal corresponding to the image is generated. Its operation control method has a feature, i.e. each image is defined for differentiation similar to an ASCII coding theory. Each code has a mating bright and dark signal which must be displayed on the screen simultaneously within a set duration. For instance, flickering a bright and dark signal eight times within one second. Such a method has drawbacks, e.g., the set duration of each cycle is too short, and to do decoding and comparison of the maximum similarity value is quite difficult; moreover, in the event that the flickering signals of two neighboring images differ only by one time of brightness and darkness, the difference is too small to identify different stimuli; hence users have to focus constantly to watch the flickering signal until the maximum similarity value exceeding the threshold. It creates a great spiritual and time burden to the users. Moreover, if two neighboring images have different flickering signals users' observation could be interfered and result in erroneous analysis and induce a low accuracy.

A fourth type of brainwave control method is based on event related potential signals (ERPs). A peak value will appear in brain electrical signal in 300 ms after accumulating 10 times of brain signals of stimuli of the same object. For separating different stimuli of objects, it requires that the time interval between two stimuli to be more than 1 second, so as to ensure that the response of the subject to the proceeding stimuli has ended or disappeared before the arrival of a new stimulus. Thus, it requires a long period of time (exceeds 50 seconds for five objects' control) to identify the meaning of ERPs among different objects and sends out a control signal. The ERPs cannot be used for real-time control.

### SUMMARY OF THE INVENTION

The primary object of the present invention is to overcome the disadvantages of the conventional non-contact operation control systems that have greater constraints, low accuracy and a long processing time

To achieve the foregoing object, the present invention provides a brainwave control system and method operable through time differential event-related potential. The system includes a brainwave capturing unit, a brainwave signal processing apparatus and a display unit. The brainwave capturing unit is attached to a user's head to capture user's brainwave signals. The brainwave signal processing apparatus includes at least one signal processor and one central processor. The signal processor is connected to the brainwave capturing unit to receive user's brainwave signals, amplify the brainwave signals and remove noises and perform filtering process, then convert to digital signals. The central processor receives the digital signals and performs analysis and processing, and executes commands according to identification results. The display unit is a display screen connected to the central processor to display at least two sets of stimuli which correspond to different control commands. The stimuli can be positioned around a controlled targeted and moved with therewith or on a fixed location on the display screen.

The method of the present invention includes the following steps: a. the central processor repeatedly displays at least two sets of stimuli in a randomly flickered fashion according to a set short time difference (e.g., 0.1 second) on the display unit to give a user sensory stimulation and evoke time differential brainwave signals i.e. time differential event related potential signals; b. the brainwave capturing unit captures and receives user's brainwave signals; c. the signal processor amplifies the brainwave signals, removes noises and performs filtering process, and converts analog signals to digital signals sent to the central processor; and d. the central processor receives the and accumulates the digital signals, and performs analysis and processing to generate identification results, and executes control commands accordingly.

The invention thus formed provides many advantages, notably:

1. By short time interval stimuli, the user generates time differential event related potential signals. By analyzing the maximal difference between two peaks within predefined time periods (e.g. 150˜200 ms and 250˜300 ms), the target stimulus can be determined. Thereafter, there is no need to do analysis and comparison for similarity. The control command can be executed rapidly and accurately to meet user's requirements of operation control.

2. The number of stimuli of each object can be adjusted for increasing or decreasing according to actual requirements. And user needs only to watch and count in mind the target image flickering times without caring of blinking or the eyeballs watching squarely the screen, the burden in use is alleviated. The invention not only is applicable for general users, but also provides a convenient and practical non-contact operation control method for invalid people with handicapped limbs. It offers greater usability and practicality.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 shows the time differential event related potential signals for 4 targets' control.

FIG. 2 is a schematic structure of the invention.

FIG. 3 is an operational diagram the invention.

FIG. 4 is a process flowchart of the invention.

FIGS. 5 and 6 are schematic views of an embodiment of the invention in use conditions.

### DETAILED DESCRIPTION OF THE PREFERRED EMBODIMENT

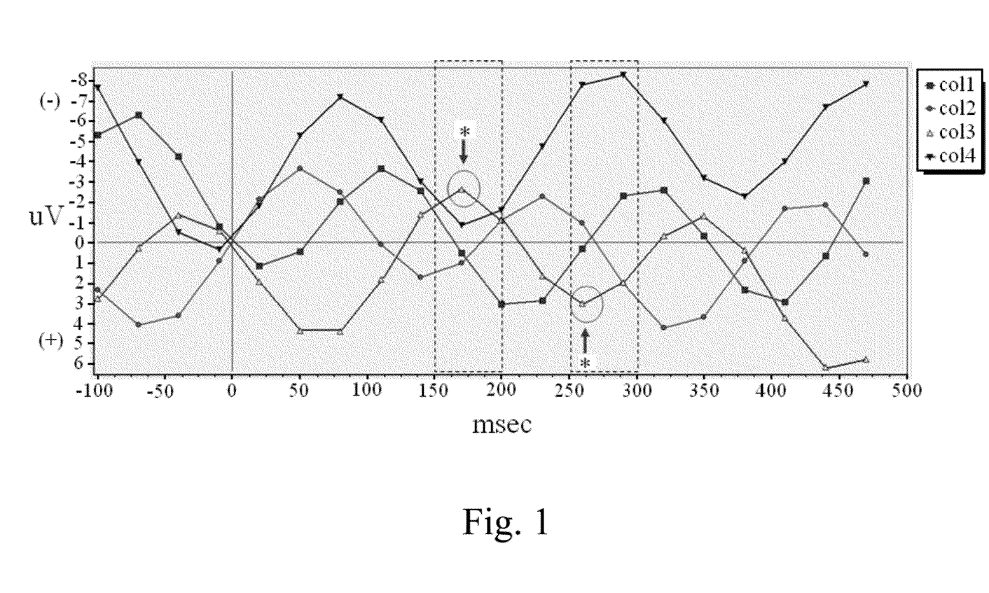

The basic of the present invention is time differential event related potential. So-called time differential event related potential means that the stimuli are generated by a short time interval (e.g., 0.1 second) and the generates event related potentials with different latency time. Since user only gives attention on the target stimulus, the peaks of brainwave within predefined time periods can be use to represent his/her requirement (see FIG. 1). For example, the target stimulus has the maximal difference between two peaks within 250˜300 ms and 150˜200 ms, i.e. the maximal difference is equal to the peak value within 250˜300 ms minus the peak value within 150˜200 ms. In this way, the system can meet user's operation control requirements correctly and rapidly (less than 2 seconds).

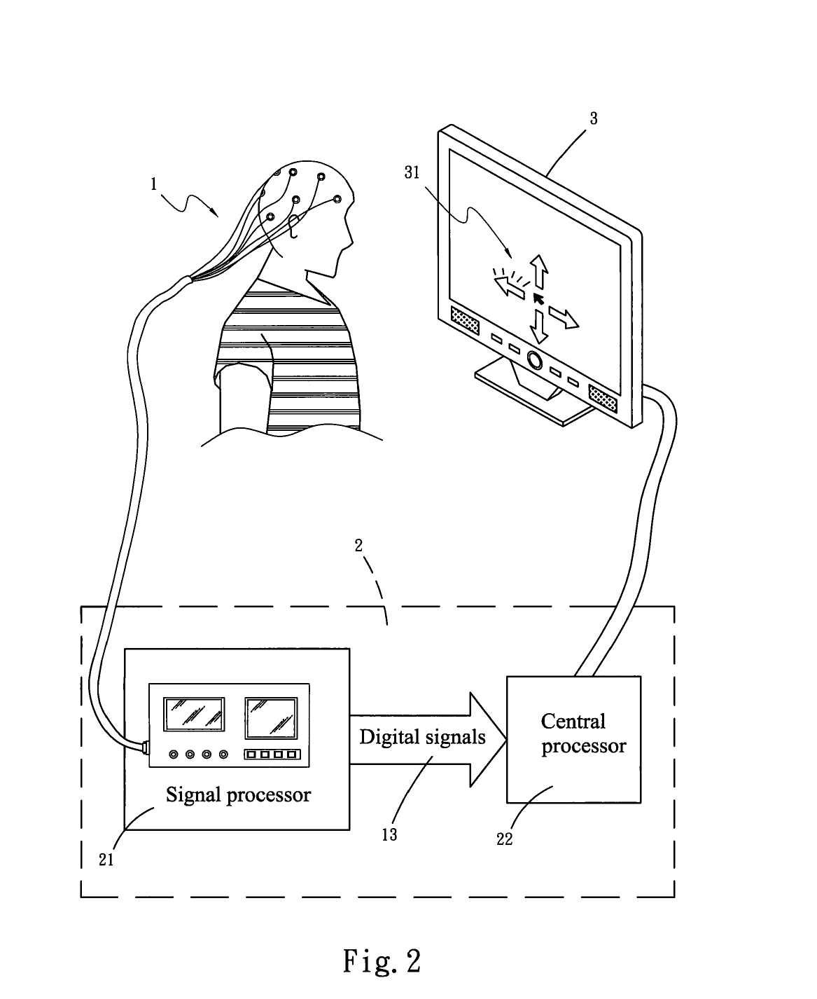

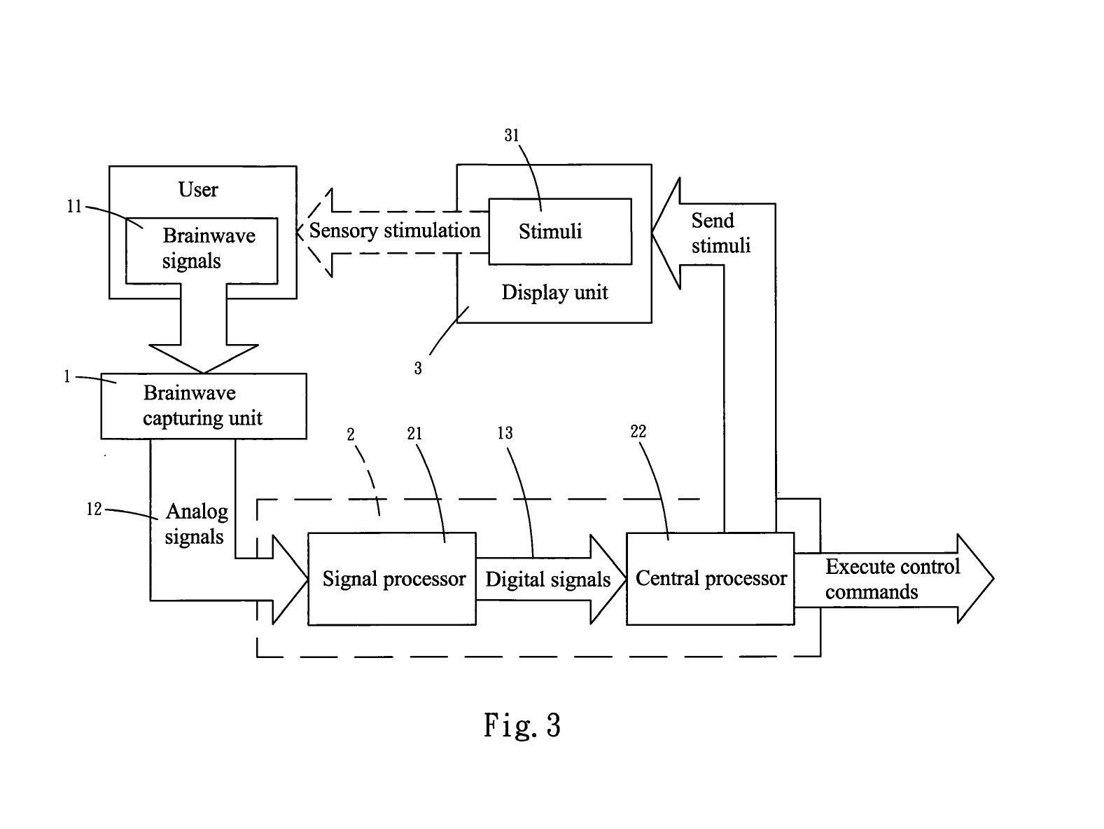

Please referring to FIGS. 2 and 3, the present invention aims to provide a brainwave control system operable through time differential event-related potential. The system includes a brainwave capturing unit 1, a brainwave signal processing apparatus 2 and a display unit 3.

The brainwave capturing unit 1 is attached to a user's head to capture user's brainwave signals 11. It can be a plurality of brainwave electrode patches or a brainwave gathering cap fabricated by plating of silver and silver compound.

The brainwave signal processing apparatus 2 includes at least one signal processor 21 and one central processor 22. The signal processor 21 is connected to the brainwave capturing unit 1 to receive user's brainwave signals 11, and amplify the signals, remove noises and perform filtering process, then convert analog signals 12 to digital signals 13. The central processor 22 receives the digital signals 13, and performs analysis and processing, generates identification results and executes commands accordingly.

The display unit 3 is a display screen connected to the central processor 22 to display at least two sets of stimuli 31 generated by the central processor 22. The stimuli 31 correspond to different control commands, and can be positioned around a controlled targeted and moved therewith or on a fixed location on the display screen.

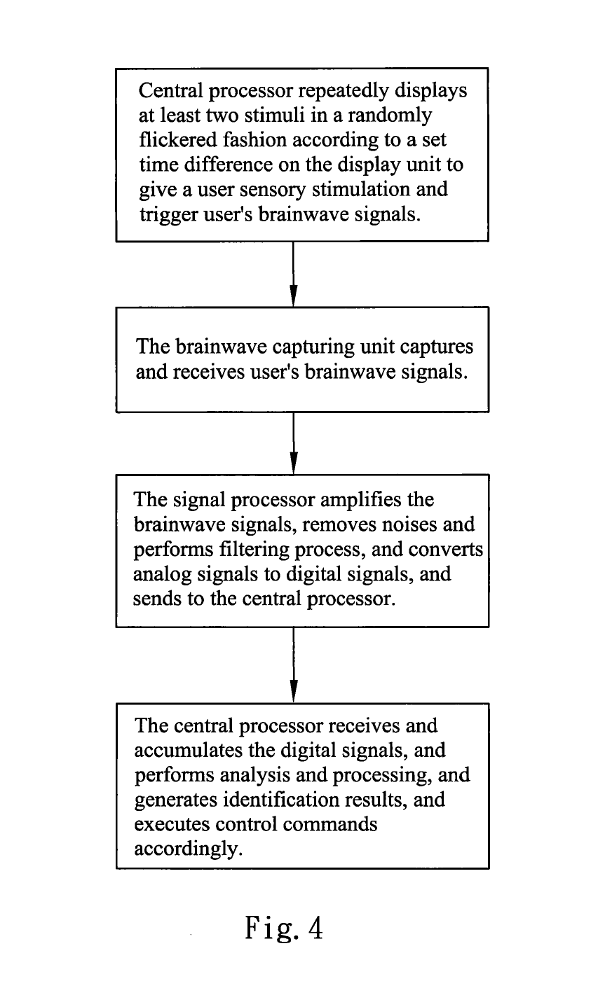

Referring to FIGS. 3 and 4, the invention also provides a method to perform brainwave control through time differential event-related potential. The method includes the following steps:

a. The central processor 22 repeatedly displays at least two sets of stimuli 31 in randomly flickered fashion according to a set short time difference on the display unit 3 to give a user sensory stimulation and trigger user's brainwave signals 11;

b. the brainwave capturing unit 1 captures and receives user's brainwave signals 11;

c. the signal processor 21 amplifies the brainwave signals 11, removes noises and performs filtering process, and converts analog signals 12 to digital signals 13 sent to the central processor 22; and

d. the central processor 22 receives and accumulates the digital signals 13, and performs analysis to find the maximal difference between two peaks within predefined time periods (e.g. 150˜200 ms and 250˜300 ms) and processing to generate identification results, and executes control commands accordingly.

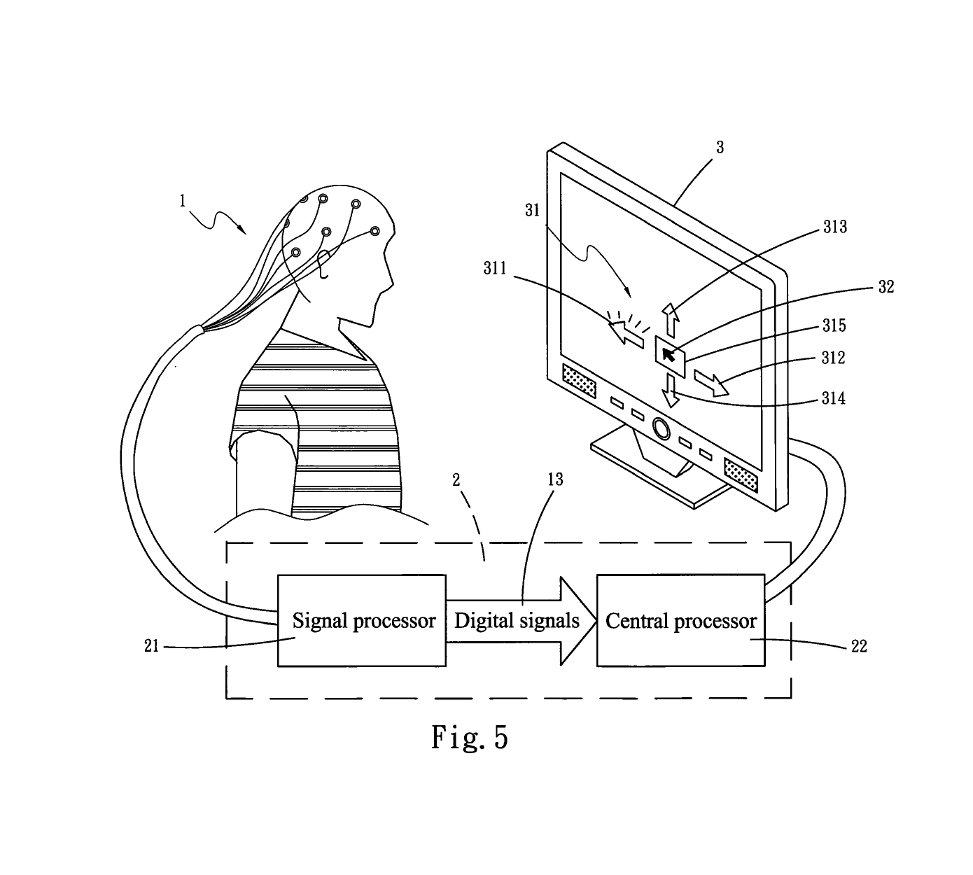

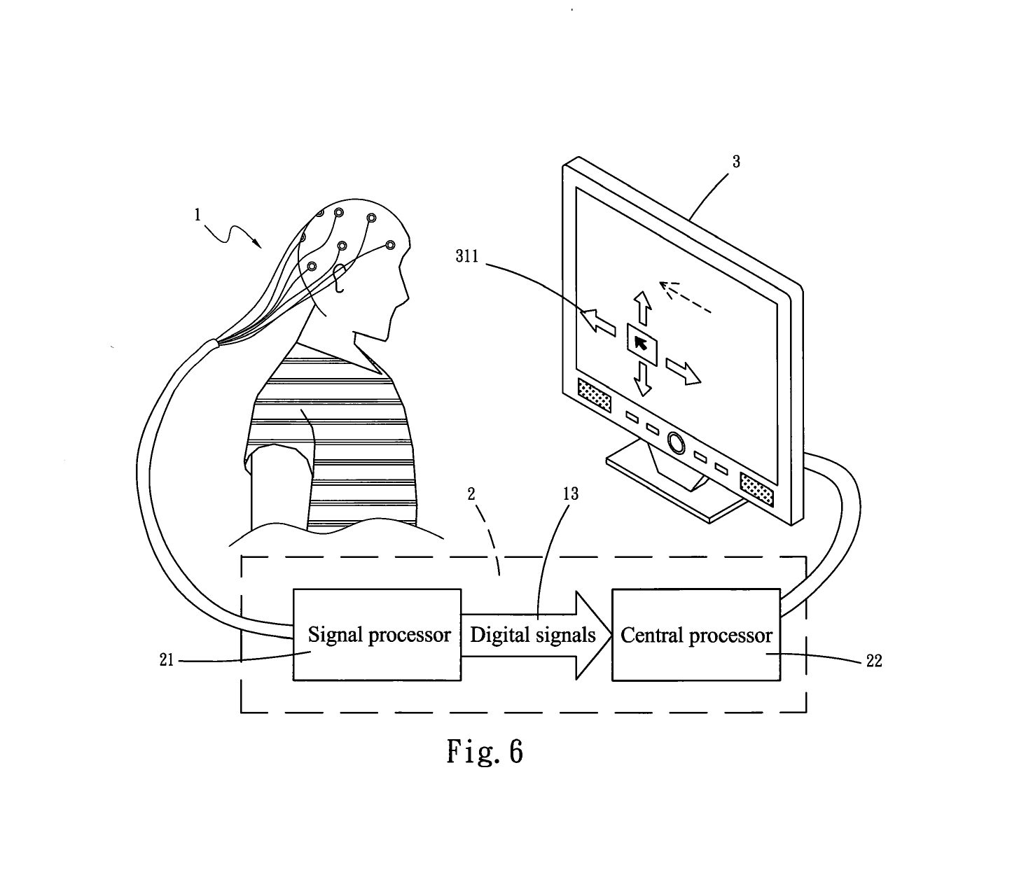

Please refer to FIGS. 3, 5 and 6 for an embodiment of the invention. The display unit 3 is a display screen connected to the central processor 22, and displays five stimuli 31, including a leftward symbol 311, a rightward symbol 312, an upward symbol 313, a downward symbol 314 and a stop symbol 315, to correspond to a controlled target. In this embodiment a mouse cursor 32 is the controlled target. The stimulus 31 surround the mouse cursor 32 and correspond to the mouse cursor 32 to execute leftward movement, rightward movement, upward movement, downward movement and movement stop operations. The central processor 22 repeatedly displays the five stimuli 31 in a randomly flickered fashion according to a set short time difference on the display unit 3 to give a user sensory stimulation and trigger user's brainwave signals 11. For instance, if the user wants to move the mouse cursor 32 leftwards, he/she focuses on the leftward symbol 311, such as counts in mind the number of flickering so that the brain generates an time differential event-related potential of the leftward symbol 311 stimulation and the peaks of brainwave within predefined time periods can be use to represent his/her requirement.

Next, the brainwave capturing unit 1 captures and receives user's brainwave signals 11; the signal processor 22 amplifies the brainwave signals 11, removes noises and performs filtering process, and converts analog signals 12 to digital signals 13 sent to the central processor 22.

The central processor 22 receives and accumulates the digital signals 13, and performs analysis and processing to generate recognition results to confirm user's selection of the stimuli 31 being the leftward symbol 311, then controls the mouse cursor 32 to execute leftward movement to meet user's requirement. When the mouse cursor 32 is controlled and moved, the display positions of the stimuli 31 also move with the movement of the mouse cursor 32 in a friendly way to enhance usability of control operation.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

1. A brainwave control system operable through time differential event-related potential, comprising:
a brainwave capturing unit attached to a user's head to capture user's brainwave signals;
a brainwave signal processing apparatus including a signal processor and a central processor, the signal processor being connected to the brainwave capturing unit to receive user's brainwave signals, amplify the signals and remove noises and perform filtering process, then convert analog signals to digital signals, the central processor receiving the digital signals and performing analysis and processing, and generating identification results and executing control commands accordingly; and
a display unit which is a display screen connected to the central processor to display a least two sets of stimuli generated by the central processor, the stimuli corresponding to varying control commands.

2. The brainwave control system of claim 1, wherein the stimuli are positioned around a controlled target and moved therewith or on a fixed location on the display screen.

3. The brainwave control system of claim 1, wherein the brainwave capturing unit is a plurality of brainwave electrode patches plated with silver or silver compound or a brainwave gathering cap.

4. A brainwave control method operable through time differential event-related potential, comprising the steps of
a. displaying repeatedly at least two sets of stimuli in a randomly flickered fashion according to a set short time difference on a display unit through a central processor to give a user sensory stimulation and trigger user's brainwave signals;
b. capturing and receiving user's brainwave signals via a brainwave capturing unit;
c. amplifying the brainwave signals through a signal processor which also removes noises and performs filtering process, and converts analog signals to digital signals sent to the central processor; and
d. receiving and accumulating the digital signals via the central processor which also performs analysis to find the maximal difference between two peaks within predefined time periods and processing to generate identification results, and executes control commands accordingly.

5. A brainwave control method of claim 4, wherein the predefined time periods are 150˜200 ms and 250˜300 ms.
